(19) United States
(12) Patent Application Publication
(10) Pub. No.: US 2022/0397378 A1
(43) Pub. Date: Dec. 15, 2022

(54) MULTIFUNCTIONAL MORPHOLOGICAL MEASURING APPARATUS

(71) Applicant: Yannick KENGA, Houston, TX (US)

(72) Inventor: Yannick KENGA, Houston, TX (US)

(21) Appl. No.: 17/343,752

(22) Filed: Jun. 10, 2021

(51) Int. Cl. G01B 3/1005; A41H 1/02; G01B 3/1003; G01B 5/02; G01B 3/1094; A61B 5/107

(52) U.S. Cl. CPC G01B 3/1005; A41H 1/02; G01B 3/1003; G01B 5/025; G01B 3/1094; A61B 5/107; G01B 2003/1079

(57) ABSTRACT

This multifunctional morphological measuring apparatus comprising a housing (104) having a slot, and a graduated flexible tape measure (6) inserted into said slot and having a first end held in the housing (104) and a second end (105) disposed at the exterior of the housing (104), the housing (104) enclosing means for winding the tape (6) in said housing (104) and an electronic control system (7) comprising an electronic card (15) having a computer and electronic memory components, the apparatus (100) further comprising means for measuring (12) a morphological index characteristic of a part of the human body (U0) around which the tape is at least partially wound (6).

### FIELD OF THE INVENTION

The present invention relates to measuring devices allowing a user to measure morphological curvatures, in particular circumferences (torso, arms, thighs).

The present invention aims to constitute a multifunction and connected measuring device which allows measurements of the morphology and the fat mass constitution of the anatomy of the user, while collecting the measured data.

### STATE OF THE PRIOR ART

Measuring systems are known which make it possible to measure a length of curvature, in particular of muscles. None of these systems is connected for the collection of measurements.

Above all, the existing devices do not make it possible to combine a curvature length measurement and a body characteristic measurement, such as the fat mass index or the body mass index.

### SUMMARY OF THE INVENTION

The object of the invention is to overcome at least some of the aforementioned drawbacks and to propose an apparatus capable of combining advantages of compactness, precision and simplicity for its implementation.

In view of the foregoing, the invention relates to a multifunction morphological measuring device comprising a housing having a slot, and a graduated flexible measuring tape inserted into said slot and having a first end held in the housing and a second end arranged outside the housing, the housing enclosing means for winding the tape in said housing and an electronic control system comprising an electronic card having a computer and electronic memory components, the apparatus further comprising measuring means a morphological index characteristic of a part of the human body around which the tape is at least partially wound.

Advantageously, one of the electrodes is mechanically linked to the housing or to the electronic control system.

According to one embodiment, the electronic control system comprises a transmitter for telemetric transmission of data measured by the morphological index measuring means.

In one embodiment, one of the means for measuring the characteristic morphological index of a part of the human body around which the tape is at least partially wound is offset from the housing by an electric cable.

Advantageously, an electric charge accumulator and a power supply port.

The multifunction morphological measuring device may further include a control button interfacing the user and the electronic control system.

Preferably, the morphological index measuring means comprise a rotary transducer which measures the length of the unwinding of the tape from the casing and encodes it into an electrical quantity intended for the electronic control system.

The invention also relates to a multifunction morphological measuring device in which the electrodes are adapted to measure a morphological index characteristic of a part of a human body from a difference in electric potential between its two terminals formed by the pair of electrodes, applied around said part of human body.

### DETAILED DESCRIPTION OF THE FIGURES

The invention will be better understood from a detailed study of a few embodiments taken by way of nonlimiting examples and illustrated by the appended drawings, in which:

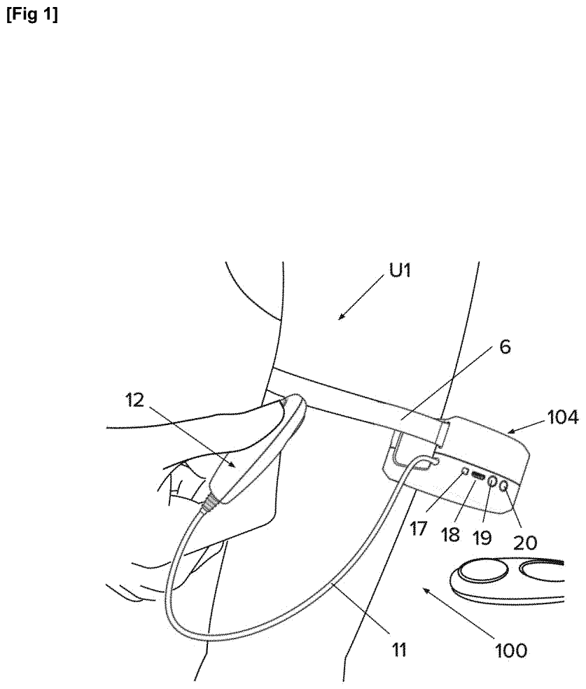

FIG. 1 shows a measuring device according to a first particular embodiment and placed on the arm of a user;

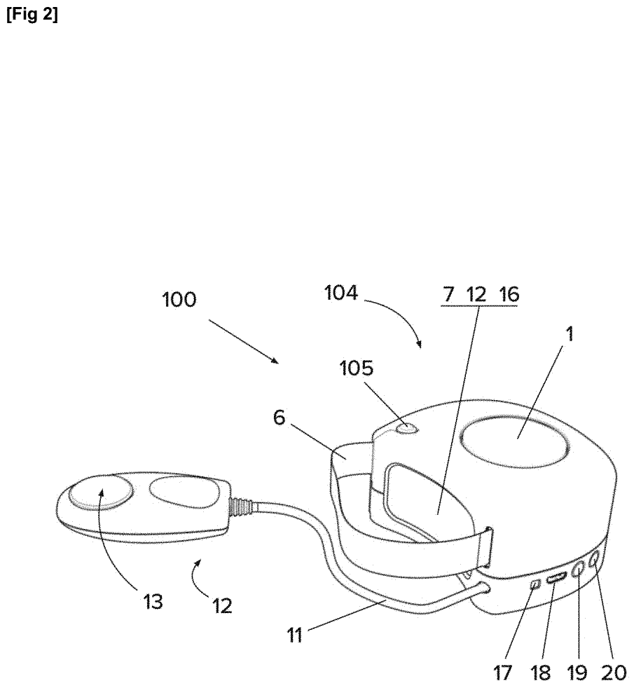

FIG. 2 shows a perspective view of the apparatus according to the first embodiment;

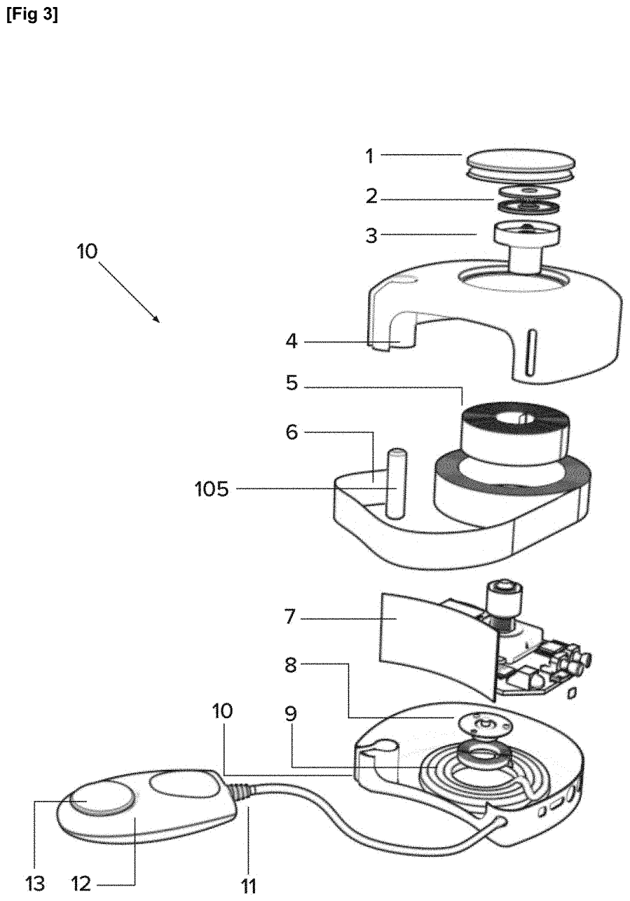

FIG. 3 shows an exploded view of the same embodiment of the apparatus;

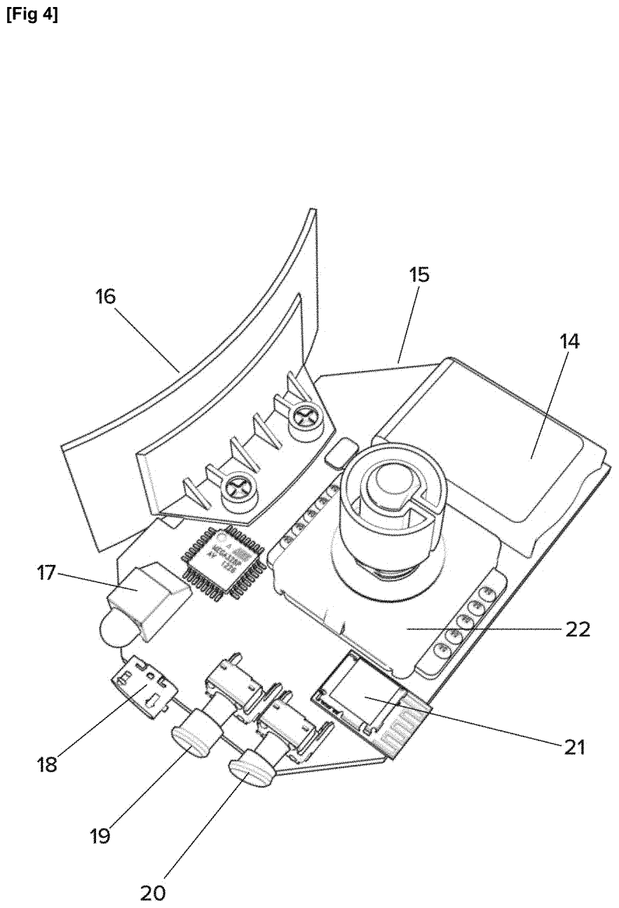

FIG. 4 details the electronic control device of the device;

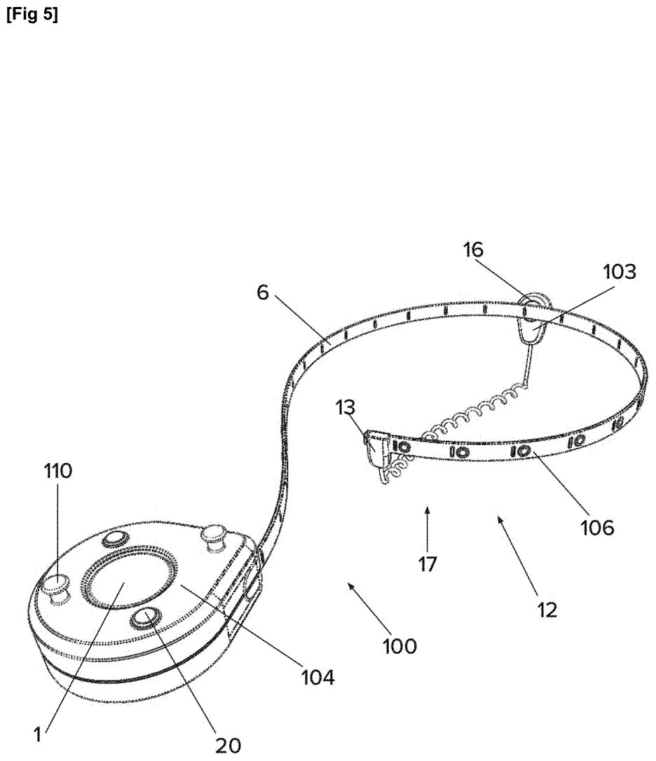

FIG. 5 shows a second embodiment of the apparatus; and

### DETAILED DESCRIPTION OF THE FIGURES

FIG. 1 illustrates the multifunction morphological measuring device 100 of the invention, placed in the measurement position on the arm U0 of a user.

FIGS. 2 and 3 detail the contents of the device 100.

The device can be used on many parts of the human body U0, including the arms and legs.

The apparatus 100 comprises a housing 104 similar to that of the measuring tape measures known from the prior art. Housing 104 has a slot through which a graduated flexible tape measure 6 passes for direct visual measurements. The tape 6 has a first end held in the housing 104 and a second end 105 disposed outside the housing 104. It can be made at least partially of fiberglass.

The housing 104 is composed, for example, of an upper housing 4 made of acrylonitrile butadiene styrene assembled on a lower housing 10 made of polyurethane.

The manufacture of the housing 104 is similar to that of a standard tape measure, with a housing made by injection molded enclosing the retractable tape 6, which has graduations or indications at regular distances 106.

The housing 104 contains means for winding the tape 6 in said housing 104. The means for winding the tape 6 comprise, for example, winding elements actuated mechanically by elastic return or by spring effect against the unwinding exerted by user, such as one or more steel torsion springs 5, 9 as shown in FIG. 3.

The support 3 has in particular a role of bearing which prevents the upper button 1 from rotating on itself with the winding or unwinding of the tape 6. It can for this purpose be supplemented with two serrated plates 2 for a perfect dissociation of the tape, rotation of the coil springs 5, 9 and of the upper knob 1.

The springs 5, 9 advantageously comprise a flat coil spring for an activation coil of the button 1, which allows the overall size of the apparatus 100 to be minimized.

The housing 104 may include a groove provided for holding the second end 105 of the tape 6 in position.

Thus, the tape 6 can form a loop when it is unwound, through which the user can position a part of his body U0 to be analyzed. The tape winding means 6 ensure the support of the tape 6 as close as possible to the part U0.

The housing 104 further contains an electronic control system 7 comprising a printed circuit on an electronic card 15, for example made of copper and having a computer and electronic memory components, for example a RAM memory. The computer comprises for example a microprocessor. It is possible to use the computer of a remote device, in particular of a multifunction telephone.

The electronic system 7 may include a power cable or an electric charge accumulator 14 and a power supply port 18, for example of the standard USB or micro-USB type, for example made of stainless steel, for electrically supplying the accumulator 14.

The accumulator 14 may include a rechargeable battery, in particular a polymer battery of the 350 mAh lithium-ion type, for prolonged use without recharging. The accumulator 14 can also be removable or consist of standard batteries.

The apparatus 100 further comprises means 12 for measuring a morphological index characteristic of a part of the human body U0 around which the tape 6 is at least partially wound. The morphological index is for example the mass index. body weight (BMI), fat mass or muscle mass of the U0 body part analyzed. Thus, the device 100 uses techniques for calculating the BMI to determine the level of body fat and muscle gain in localized areas of the body U0, in particular areas susceptible to sports training.

The morphological index measuring means 12 comprise at least one pair of complementary electrodes 13, 16, called positive electrode and negative electrode.

At least one electrode of the pair of electrodes 13,16 is arranged on an inner face of the strip 6. It is intended to come into direct contact with the skin of the U0 part analyzed.

As illustrated in FIGS. 1 to 3, one of the electrodes 13,16 can be mechanically linked to the housing 104 or to the electronic control system 7.

As illustrated in FIG. 5, an electrode 13 can be positioned in a fixed or removable manner on the outer end 105 of the strip 6. The second electrode 16 is then intended to be placed on the strip, for example by a removable magnet attachment or clipping onto one of the regular elements 106 of the strip 6. The two electrodes are also then electrically connected to one another by an extensible cord for measuring their potential difference.

The housing 104 may include storage elements 110 for said cord.

The electrodes are made of a conductive material, in particular metal, in particular based on aluminum.

The electrodes 13,16 are suitable for measuring the characteristic morphological index of a human body part U0 from a difference in electric potential between its two terminals formed by the pair of electrodes 13,16 applied around said part. of human body U0.

They can detect the difference in potential or electric voltage between the two electrode terminals 13, 16 and send it to the computer which is configured to deduce the characteristic index from a correspondence table of voltage data and d morphological index. For example, the device 100 makes it possible to evaluate a fat mass index of a biceps, which will decrease if the person is gaining muscle.

A characteristic measurement of the morphological state of a muscle of the person is thus deduced therefrom, which can be recorded and correlated with measurements taken repeatedly to monitor the effectiveness of a training or a diet.

The device 100 is particularly practical for its adaptability to different morphologies.

The electronic system 7 may further include a transmitter for telemetric transmission of data 21 measured by the morphological index measuring means 12.

The transmitter 21 can for example upload the data measured and stored in memory on a remote server or towards a device such as a multifunction cellular telephone. It can conventionally use for this purpose telecommunication waves by radio frequency or by Bluetooth® for example.

It may include in addition a Bluetooth pairing button 20 of the device 100 to a software application which processes the data coming from the device 100, that is to say recovers, records and analyzes them, then communicates, the data processed to the user. For example, the device 100 works by treating the electrical resistance to a low electrical current as it passes over a part of the body U0 between the electrodes, at least one electrode of which is coupled to the electronic system 7 and relay this measured current to a microprocessor for analysis.

One of the means 12 for measuring the characteristic morphological index of a part of the human body U0 around which the tape 6 is at least partially wound can be offset from the housing 104 by an electric cable 11. It is then on a lever 12 as illustrated.

The handle 12 may for this purpose include one of the electrodes 13, 16 and an activation button 19. The button 19 of the handle 12 can then be positioned and held on the opposite side of the body part U0, directing the two the electrodes 13, 16 towards each other.

The morphological index measuring means 12 may comprise a rotary transducer 22 which measures, as a morphological index, the length of the unwinding of the tape 6 out of the housing 104, in order to measure the part U0. The transducer 22 encodes the measurement of unwinding of the tape 6 into an electrical quantity intended for the electronic control system 7. A rotary encoder is for example used to measure the circumference of the part of the body U0, at the same time to simultaneously follow the physical growth, and to better inform the analysis of the morphological index.

The apparatus 104 may further include a control button 19 interfacing the user and the electronic control system 7, to control its ignition, to control the taking of measurements.

A light-emitting diode 17 can supplement the use of the control buttons 19, 20, by giving indications to the user during the use of the device 100, by its flashing or its color. For example, LED 17 flashes blue during pairing, or indicates a low battery level in orange or red.

The control button 19 can be placed on the housing 104.

Another control button 19 can also be placed on the joystick 12.

1. Multifunctional morphological measuring apparatus (100) comprising a housing (104) having a slot, and a graduated flexible tape measure (6) inserted into said slot and having a first end held in the housing (104) and a second end (105) arranged outside the housing (104), the housing (104) enclosing means for winding the tape (6) in said housing (104) and an electronic control system (7) comprising an electronic card (15) having a computer, electronic memory components, an electric charge accumulator (14) and a power supply port (18), the apparatus (100) further comprising means for measuring (12) of a characteristic morphological index of 'a part of the human body (U0) around which the tape (6) is at least partially wound, said morphological index measuring means (12) comprising: —a rotary transducer (22) which measures the length of the unwinding of the tape (6) from the housing (104) and encodes it into an electrical quantity intended for the control system electronic (7)
a lever which is offset from the housing (104) by an electric cable (11) and which has an activation button (19) interfacing the user and the electronic control system (7),
at least one pair of complementary electrodes (13,16) of metallic conductive material based on aluminum, one of said electrodes (13,16) being mechanically linked to the electronic control system (7), and one of said electrodes (13, 16) being on the joystick,
the electronic control system (7) comprising a transmitter for telemetric transmission of data (21) measured by the morphological index measuring means (12).

2. A multifunctional morphological measuring apparatus (100) according to any one of claims 2 to 4, wherein the electrodes (13,16) are adapted to measure a morphological index characteristic of a human body part (U0) from a difference in electric potential between its two terminals formed by the pair of electrodes (13,16) applied around said part of the human body (U0).
